(19) United States
(12) Patent Application Publication
(10) Pub. No.: US 2011/0008332 A1
(43) Pub. Date: Jan. 13, 2011

(54) Combination Therapy to Treat Persistent Viral Infections

(75) Inventors: David Brooks, San Diego, CA (US); Michael B.A. Oldstone, La Jolla, CA (US)

Correspondence Address: MERCK;C/O DNAX, LEGAL DEPARTMENT, 901 CALIFORNIA AVENUE, PALO ALTO, CA, 94304, US

(73) Assignee: THE SCRIPPS RESEARCH INSTITUTE, La Jolla, CA (US)

(21) Appl. No.: 12/739,637

(22) PCT Filed: Oct. 29, 2008
(86) PCT No.: PCT/US2008/081614

§ 371 Date: Sep. 7, 2010

Related U.S. Application Data
(60) Provisional application No. 60/984,103, filed on Oct. 31, 2007.

(51) Int. Cl. A61K 39/395; A61K 39/00; A61P 37/04; A61P 31/12

(52) U.S. Cl. 424/133.1; 424/184.1; 424/145.1

(57) ABSTRACT

The present invention pres ides combination therapies to treat persistent viral infections. In particular, combinations of vaccines and IL-10 or IL-10 receptor (IL-10R) antagonists to clear such infections are provided.

### FIELD OF THE INVENTION

The present invention provides combination therapies to treat persistent viral infections. In particular, combinations of vaccines and IL-10 or IL-10 receptor (IL-10R) antagonists to more efficiently clear such infections are provided.

### BACKGROUND OF THE INVENTION

Viral infections trigger robust T cell responses which are crucial to clear infections. However, in response to some viral infections, antiviral CD4 and CD8 T cells become unresponsive to viral antigens and are either physically deleted or persist in nonfunctional or “exhausted” states. This exhausted state is characterized by the inability to produce antiviral and immune-stimulatory cytokines, lyse virally infected cells, or proliferate (see, e.g., Zajac, et al. (1998) J. Exp. Med. 188:2205-2213; Gallimore, et al. (1998) J. Exp. Med. 187:1383-1393; Wherry, et al. (2003), J. Virol. 77:4911-4927; and Brooks, et al. (2006) J. Clin. Invest. 116:1675-1685).

Multiparameter loss of T cell function directly facilitates persistence, as evidenced by the fact that prolonged T cell responses strongly correlated with clearance and control of hepatitis C virus (HCV) and human immunodeficiency virus (HIV) infections in humans and lymphocytic choriomeningitis virus (LCMV) infection in mice (see, e.g., Barber (2006) Nature 439:682-687: Brooks, et al. (2006) Nat. Med. 12:1.301-1309; Ejmaes, et al. (2006) J. Exp. Med. 203:2461-2471; Oldstone et al. (1986) Nature 321:239-243; Thimme, et al. (2001) J. Exp. Med. 194:1395-1405; Gandhi and Walker (2002) Ann. Rev. Med. 53:1.49-172; and Shoukry, et al. (2004) Ann. Rev. Microbiol. 58:391-424.). To date, vaccines to report antiviral T cell activity and control persistent viral infections have only been marginally successful (see, e.g., Autran, et al. (2004) Science 305:205-208).

Recently, based upon genetic deletion and antibody blockade studies, IL-10 was identified as a single dominant factor that determines whether a viral infection is cleared or persists (see. e.g., Brooks et al. (2006) supra; and Ejmaes, et al. (2006) supra). Antibody blockade of IL-10 during an established persistent infection and following T cell exhaustion, restored T cell function leading to enhanced clearance of virus. During persistent viral infection. Programmed Death-1:Programmed Death-Ligand 1 (PD-1:PD-L1) interactions further limit T cell function and antibody blockade of PD-L1 can stimulate T cell activity (see, e.g., Barber, et al. (2005) supra). One proposed strategy is dual antagonism of the IL-10 and PD-1 pathways to clear persistent viral infections (see, e.g., Marinic and von Herrath (2008) Trends Immunol. 23:116-124).

Because increased IL-10 expression is observed during many persistent viral infections in humans (HIV, HCV, HBV, for example) and is directly correlated with decreased T cell responsiveness and the failure to control viral replication. Therefore a need exists to better control IL-10 activity in persistent viral infection. The present invention fulfills this need by providing combination therapies using IL-10 antagonists to treat such infections.

### BRIEF DESCRIPTION OF THE DRAWINGS

FIG. 1a-1d shows IL-10R blockade enables effective stimulation of antiviral T cell responses by therapeutic vaccination. FIG. 1a depicts a schematic representation of anti-IL-10R antibody treatment and DNA vaccination. LCMV-CI 13 infected mice were either left untreated, treated with DNA vaccine (encoding the entire LCMV-GP), treated with an anti-IL-10R blocking antibody or co-treated with DNA vaccine phis anti-IL-10R antibody. Anti-IL-10R treatment was initiated on day 25 after infection and continued every 2-3 days for 2 weeks. DNA vaccination was administered on day 29 and day 34 after infection. T cell responses were then analyzed on day 39 after infection. FIG. 1b shows cytokine production as quantified by ex vivo peptide stimulation and intracellular staining. Flow plots illustrate the frequency of IFNγ-producing LCMV-GP₃₃₋₄₁ specific CD8 T cells. Data are representative of 4 mice per group. FIG. 1c indicates the number of IFNγ-producing LCMV-GP₃₃₋₄₁. GP₂₇₆₋₂₈₆ and NP₃₉₆₋₄₀₄ specific CD8 T cells following each treatment regimen. Circles represent individual mice *, p<0.05 compared to untreated and DNA vaccination alone. p<0.05 compared to ail other treatment groups. FIG. 1d T represents the average fold increase in the number of TNFα producing LCMV-GP₃₃₋₄₁ specific CD8 T cells in each treatment group compared to isotype treated (which is set to 1) and are the average±standard deviation (SD) of 3 experiments containing 4-6 mice. *, p<0.05 compared to untreated and DNA vaccination alone. **, p<0.05 compared to all other treatment groups.

FIG. 2a-2c shows that CD4 T cell responses are enhanced by IL-10R blockade and vaccination. FIG. 2a shows LCMV-CI 13 infected mice were treated and analyzed as described in FIG. 1. The frequency of cytokine producing LCMV-GP₆₁₋₈₀ specific CD4 T cells was quantified by ex vivo peptide stimulation and intracellular staining. Flow plots illustrate the frequency of IFNγ-producing LCMV-GP₆₁₋₈₀ specific CD4 T cells following each treatment (day 39 post infection). Data are representative of 4 mice per group. FIG. 2b illustrates the number of IFNγ-producing LCMV-GP₆₁₋₈₀ specific CD4 T cells and quantified as described in FIG. 2B. *, p<0.05 compared to untreated and DNA vaccination alone. **, p<0.05 compared to all other treatment groups. FIG. 2c shows the average fold increase in the number of IL-2 producing LCMV-GP₆₁₋₈₀ specific CD4 T cells following each treatment regimen compared to isotype treatment. Individual bars represent the average value ±SD of 3 experiments containing for 4-6 mice per group. *, p<0.05 compared to untreated and DNA vaccination alone. **, p<0.05 compared to all other treatment groups.

FIG. 3a and 3b show IL-10R blockade combined with vaccination restores T cell function. Prior to infection mice were seeded with LCMV-specific TcR tg (FIG. 3a) CD8+ (P14) and (FIG. 3b) CD4+ (SMARTA) T cells and then infected with LCMV-CI 13. Mice were treated with isotype control antibody, anti-IL-10R blocking antibody and/or DNA vaccine as described in FIG. 1a. Bar graphs indicate the fold increase in the number of P14 and SMARTA cells on day 39 after infection. Individual bars represent the average ±SD of 5 mice per group. *, significant (p<0.05) increase in the average number versus isotype and DNA vaccine alone. **, significant (p<0.05) increase versus all other conditions.

FIG. 4a-4b shows accelerated control, of persistent viral Infection by alleviating the immunosuppressive environment and stimulating T cell responses. FIG. 4a shows LCMV-CI 13 infected mice were infected and treated as described in FIG. 1a. The bar graph illustrates the fold decrease in serum virus titers in response to each treatment regimen. Fold decrease was determined by dividing the virus titers in each mouse on day 25 post infection (i.e., prior to treatment) by the virus titer on day 33 after infection (i.e., during therapy). Bars represent the average ±SD of 3 mice per group and are representative of 3 separate experiments with 3-6 mice per group. FIG. 4b shows that serum viral titers were quantified on day 25 (grey circles) and day 40 (white circles) after infection (following the completion of all therapeutic treatments). Each circle represents a single mouse and the graph contains data from 3 experiments. The dashed line indicates the level, of detection of the assay (200 PFU/ml), *, indicates a significant (p<0.05) decrease in virus titers on day 40 after infection compared to untreated and DNA vaccination alone. **, virus titers are significantly (p<0.05) decreased on day 40 compared to all other treatment groups. The numbers above each group indicate the average fold decrease in virus titers between day 25 and day 40 post infection.

FIG. 5A shows the frequency of Thy1.1+ tg virus specific CD8 T cells (P14 cells) in the spleen, on day 40 after LCMV CI 13 infection, and the following treatments; a) isotype control antibody: b) anti-IL-10R blocking antibody alone; c) anti-PD-L1 blocking antibody alone; or 4) co-treated with anti-IL-10R and anti-PD-L1. blocking antibodies. Treatment was initiated on day 25 after infection and administered every 3 days for a total of 5 treatments. The graph on the right illustrates the number of P14 calls following each treatment regimen.

FIG. 5B illustrates the percentage of IFNγ and TNFα producing P14 cells in the spleen, on day 40 after LCMV CI 13 infection. The bar graph represents the average fold increase ±SD (*; p< 0.05) in the number of TNFα producing P14 cells in each treatment group compared to isotype treatment (which is set at 1). Data from FIGS. 5A and 5B are representative of 4-5 mice per treatment group and 2 experiments.

FIG. 6A shows the elimination of persistent viral infection following dual IL-10R/PD-L1 blockage. LCMV CI 13 mice were treated with the indicated antibodies and serum viral titers were quantified on day 25 (dark circles) prior to treatment, and day 40 (white circles), which was 3 days after treatment cessation. Each circle represents a single mouse within each group and graph contains data from 3 experiments. The dashed line indicates the level of detection of the assay (200 plaque forming units (PFUs)/mL serum). The number above each group represents the average fold decrease in viral titer between day 25 and day 40 (* is a decrease (p<0.05) in viral titers compared to isotype treatment; ** is a decrease (p,0.05) in viral titers compared to ail other treatment groups).

FIG. 6B shows liver viral titers on day 40 after LCMV CI 13 infection following the treatment regimes described above.

FIG. 7 shows a quantitative measurement of the total number of LCMV-GP₃₃₋₄₁ and LCMV-GP₂₇₆₋₂₈₆ tetramer positive CD8+ T cells are present in the indicated tissues. (*—p<0.05; **—p<0.01; and ***—p<0.001)

### SUMMARY OF THE INVENTION

The present invention is based, in part, upon the surprising data that neutralization of IL-10 activity in combination with vaccines synergistically combine to significantly stimulate antiviral CD4 and CD8 T cell responses and control viral replication. Further, neutralization of both IL-10 and PD-1/PD-L1 also showed a synergistic effect in the lowering of viral load in a persistent viral infection model.

The present invention provides a method of treating a chronic or persistent viral infection comprising administering to a subject in need of treatment, an effective amount of a vaccine against a virus, wherein the virus is a causative agent for the persistent or chronic viral infection, in combination with an antagonist of an immunosuppressive cytokine. In one embodiment, the combination of the vaccine against the virus and the antagonist of the immunosuppressive cytokine exhibits synergy in the treatment of the chronic or persistent viral infection. In a further embodiment, the immunosuppressive cytokine is IL-10. Additionally, the antagonist of the immunosuppressive cytokine comprises a soluble IL-10 receptor (IL-10R) polypeptide. In certain embodiments, the soluble IL-10R polypeptide comprises a heterologous polypeptide, including an Fc portion of an antibody molecule, or is pegylated. In another embodiment, the immunosuppressive cytokine is a neutralizing IL-10 or IL-10 receptor (IL-10R) antibody or antibody fragment thereof. The neutralizing IL-10 or IL-10R antibody can be a monoclonal antibody, including a humanized or fully human antibody. In further embodiment, the antibody fragment is selected from the group consisting of a Fab, Fab2, Fv, and single chain antibody fragment. The present invention contemplates that the chronic or persistent viral infection is selected from the group consisting of HBV, HCV, HIV, EBV, and LCMV. In one embodiment the vaccine is a DNA vaccine. In an alternate embodiment, the antagonist of the immunosuppressive cytokine is administered before the vaccine against the virus.

The present invention provides a pharmaceutical composition for use in the treatment of chronic or persistent viral infections comprising: (a) a vaccine against a virus, wherein the virus is a causative agent for the persistent or chronic viral infection and a pharmaceutically acceptable carrier; and (b) an antagonist of an immunosuppressive cytokine and a pharmaceutically acceptable carrier.

The present invention encompasses a kit comprising: (a) a vaccine against a virus, wherein the virus is a causative agent for the persistent or chronic viral infection and a pharmaceutically acceptable carrier; and (b) an antagonist, of an immunosuppressive cytokine and a pharmaceutically acceptable carrier.

The present invention provides a method of treating a chronic or persistent viral infection comprising administering to a subject in need of treatment an effective amount of a vaccine against a virus, wherein the virus is a causative agent for the persistent or chronic viral infection, in combination with a neutralizing IL-10 or IL-10R antibody or antibody fragment thereof. In particular, the combination of the vaccine against the virus and the neutralizing IL-10 or IL-10R antibody or antibody fragment thereof exhibits synergy in the treatment of the chronic or persistent viral infection. In one embodiment, the neutralizing IL-10 or IL-10R antibody is a monoclonal antibody, including a humanized or fully human antibody. In one embodiment, the vaccine is a DNA vaccine. Administration of the vaccine in combination with the neutralizing IL-10 or IL-10R antibody can result in a 2-fold increase in virus specific CD8 T cells; or a 5-fold increase of IFNγ producing virus specific T cells; when compared to vaccine or antibody treatment alone. The combination treatment can result in a 24-fold decrease of viral titer compared to pretreatment levels. In certain embodiments the neutralizing IL-10 or IL-10R antibody or antibody fragment thereof is administered before the vaccine against the virus.

The present invention encompasses a pharmaceutical composition for use in the treatment of chronic or persistent viral infections comprising: (a) a vaccine against a virus, wherein the virus is a causative agent for the persistent or chronic viral infection and a pharmaceutically acceptable carrier; and (b) a neutralizing IL-10 or IL-10R antibody or antibody fragment thereof and a pharmaceutically acceptable carrier.

Also provided by the present invention is a kit comprising; (a) a vaccine against a virus, wherein the virus is a causative agent for the persistent or chronic viral infection and a pharmaceutically acceptable carrier; and (b) a neutralizing IL-10 or IL-10R antibody or antibody fragment thereof, and a pharmaceutically acceptable carrier.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### 1. Definitions.

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Chronic viral infection” or “persistent viral infection” as used herein, is meant a viral infection of humans or other animals which is able to infect a host and reproduce within the cells of a host over a prolonged period of time-usually weeks, months or years, without proving fatal. Amongst viruses giving rise to chronic infections and which may be treated in accordance with the present invention are the human papilloma viruses (HPV), Herpes simplex and other herpes viruses, the viruses of hepatitis B and C (HBV and HCV) as well as other hepatitis viruses, the measles virus, all of which can produce important clinical diseases, and HIV. Prolonged infection may ultimately lead to the induction of disease which may be, e. g. in the case of hepatitis C virus liver cancer, fatal to the patient. Other chronic viral infections which may be treated in accordance with the present invention include Epstein Barr virus (EBV), as well as other viruses such as those which may be associated with tumors, or in the case of animals, various veterinary viral diseases, for example those of domestic pets or farmyard animals important in agriculture.

“Therapeutically effective amount”, means IL-10 antagonists and vaccines administered in a sufficient amount to show benefit to a patient. Such benefit may be at least amelioration of at least one symptom. The actual amount administered, and rate and time-course of administration, will depend on the nature and severity of what is being treated.

“Vaccine” refers to a composition (protein or vector; the latter may also be loosely termed a “DNA vaccine”, although RNA vectors can be used as well) that can be used to elicit protective immunity in a recipient, it should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition, or immune mediated immunosuppression). Efficacy can be established in animal models.

“Immunotherapy” refers to a treatment regimen based on activation of a pathogen-specific immune response. A vaccine can be one form of immunotherapy.

“Protect” is used herein to mean prevent or treat, or both, as appropriate, a viral infection in a subject, e.g., a persistent or chronic viral infection. Thus, prophylactic or therapeutic administration of the vaccine in combination with another agent, e.g., IL-10 antagonists, can protect the recipient subject from such persistent viral infections. Therapeutic administration of the vaccine or immunotherapy can protect the recipient from infection-mediated pathogenesis, e.g., to treat a disease or disorder such as an viral-associated neoplasm, viral-associated neoplasms include Hodgkin's lymphoma, endemic Burkitt's lymphoma, nasopharyngeal carcinoma, T cell lymphoma, gastric carcinoma, uterine leiomyosarcoma, and hepatocarcinomas.

As used herein, the term “polypeptide vaccine” refers to a vaccine comprising an immunogenic polypeptide from a causative agent, e.g., a virus, and, generally, an adjuvant. The term “adjuvant” refers to a compound or mixture that enhances the immune response to an antigen. An adjuvant can serve as a tissue depot that slowly releases the antigen and also as a lymphoid system activator that non-specifically enhances the immune response (Hood, et al., Immunology, Second Ed., 1984. Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, and potentially useful human adjuvants such as BCG (bacille Calmette-Guerin) and Corynebacterium parvum. An example of a preferred synthetic adjuvant is QS-21. Alternatively, or in addition, immunostimulatory proteins, as described herein, can be provided as an adjuvant or to increase the immune response to a vaccine. Preferably, the adjuvant is pharmaceutically acceptable.

The term “DNA vaccines” is used herein to refer to vaccines delivered by means of a recombinant vector. An alternative, and more descriptive term used herein is “vector vaccine” (since some potential vectors, such as retroviruses and lentiviruses are RNA viruses, and since in some instances non-viral RNA instead of DNA can be delivered to cells). Generally, the vector is administered in vivo, but ex vivo transduction of appropriate antigen presenting cells, such as dendritic cells, with administration of the transduced cells in vivo, is also contemplated. The vector systems described below are ideal for delivery of a vector for expression of an immunogenic polypeptide of the invention.

“Vector for expression in humans” as used herein means that the vector at least includes a promoter that is effective in human cells, and preferably that the vector is safe and effective in humans. Such a vector will, for example, omit extraneous genes not involved in developing immunity. If it is a viral vector, it will omit regions that permit replication and development of a robust infection, and will be engineered to avoid development of replication competence in vivo. Such vectors are preferably safe for use in humans; in a more preferred embodiment, the vector is approved by a government regulatory agency (such as the Food and Drug Administration) for clinical testing or use in humans.

The term “immunogenic polypeptide” refers to a viral protein, or a portion thereof, that is immunogenic and elicits a protective immune response when administered to an animal. Thus, a viral immunoprotective antigen need not be the entire protein. The protective immune response generally involves cellular immunity at the CD4 and/or CD8 T cell level.

The term “immunogenic” means that the polypeptide is capable of eliciting a humoral or cellular immune response, and preferably both. An immunogenic polypeptide is also antigenic. A molecule is “antigenic” when it is capable of specifically interacting with an antigen recognition molecule of the immune system, such as an immunoglobulin (antibody) or T ceil antigen receptor. An antigenic polypeptide contains an epitope of at least about five, and preferably at least about 10, amino acids. An antigenic portion of a polypeptide, also called herein the epitope, can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein) expressed by a modified gene or DNA sequence. The term “variant” may also be used to indicate a modified or altered gene, DNA sequence, enzyme, cell, etc., i.e., any kind of mutant.

The term “treat” is used herein to mean to relieve or alleviate at least one symptom of a disease in a subject.

The term “cure” or “curing” as used herein refers to substantially eliminating symptoms of a disease, disorder or condition associated with a viral infection in accordance with the art recognized standard. The term, “cured” as used herein refers to the state of being substantially free of symptoms associated with a disease, disorder or condition.

The term “subject” as used in this application means an animal with an immune system, such as aves and mammals. Mammals include canines, felines, rodents, bovines, equines, porcines, ovines, and primates. Aves include fowls, songbirds, raptors, etc. The invention is therefore useful for treating a disease, disorder or a condition associated with a viral infection in dogs, cats, mice, rats, rabbits, cows, horses, pigs, sheep, goats, apes, monkeys, chickens, turkeys, canaries, eagles, hawks, owls, and, particularly humans. Thus, the invention can be used in veterinary medicine, e.g., to treat companion animals, farm animals, laboratory animals in zoological parks, and animals in the wild. The invention is particularly desirable for human medicine applications.

The term, “combination therapy” refers to a therapy for treating viral infections, preferably chronic or persistent viral infections, e.g., HBV, HCV, HIV, EBV, and the like, which includes administration of an effective amount of a vaccine against the virus and an antagonist against an immunosuppressive cytokine. Also included is a combination of IL-10 and PD-1/PD-L1 antagonists. A combination therapy of this invention may include one or more antiviral agents. In addition, a combination, therapy of this invention can be used as a prophylactic measure in previously uninfected individuals after a possible acute exposure to a virus that is a causative agent for persistent or chronic viral infections. Examples of such prophylactic use of the compounds may include, but are not limited to, prevention of virus transmission from mother to infant and other settings where the likelihood of transmission exists, such as, for example, accidents in health care settings wherein workers are exposed to virus-containing blood products. Moreover, a combination therapy of this invention can be used as a prophylactic measure in previously uninfected individuals, but those at a high risk of exposure as either a systemic therapy or as topical microbicide in high risk individuals.

“Synergy” as used herein is the phenomenon in which the combined action of two therapeutic entities is greater than the sum of their effects individually.

The term “synergistic” refers to a combination which is more effective than the additive effects of any two or more single agents. A “synergistic effect” refers to the ability to use lower amounts or dosages of antiviral agents in a single therapy to treat or prevent viral infection. The lower doses typically result in a decreased toxicity without reduced efficacy. In addition, a synergistic effect can improve efficacy, e.g., improved antiviral activity, or avoid or reduce the extent of any viral resistance against an antiviral agent. A synergistic effect between a vaccine, or a pharmaceutically acceptable composition thereof, and an antagonist of an immunosuppressive cytokine, or a pharmaceutically acceptable compositions thereof, can be determined from conventional antiviral assays, e.g., as described below. The results of an assay can be analyzed using Chou and Talalay's combination method to obtain a Combination Index (Chou and Talalay, (1984) Adv. Enzyme Regul. 22:27-55) and ‘Dose Effect Analysis with Microcomputers’ software (Chou and Chou, 1987, Software and Manual, p 19-64. Elsevier Biosoft, Cambridge, UK). A Combination Index value of less than 1 indicates synergy, greater than 1 indicates antagonism and equal to 1 indicates an additive effect. The results of these assays can also be analyzed using the method of Pritchard and Shipman (Pritchard and Shipman (1990) Antiviral Research 14:181-206).

The term, “antiviral activity” refers to an inhibition of viral transmission to uninfected cells, inhibition of the replication of a virus, prevention of the virus from establishing itself in a host, or ameliorating or alleviating the symptoms of the disease caused by viral infection. These effects can be evidenced by a reduction in viral load or decrease in mortality and/or morbidity, which assays are described infra. An antiviral agent or drug, has antiviral activity and is useful for treating persistent or chronic viral infections alone, or as part of a multi-drug combination therapy.

“Interleukin-10” or “IL-10”, as used herein, or in a non-conjugated form, is a protein comprising two subunits noncovalently joined to form a homodimer. “IL-10 receptor 1”, “IL-10R”, or “IL-10R 1” refer to one subunit of the IL-10 receptor complex that confers specificity for IL-10, As used herein, unless otherwise indicated “interleukin-10”, “IL-10”, and IL-10R can refer to human or mouse IL-10 or IL-10R. IL-10 antibodies are described, e.g., in U.S. Pat. No. 6,239,260; humanized anti-IL-10 antibodies are described, e.g., in US 2005/0101770; IL-10R polypeptides are described, e.g., in U.S. Pat. No. 5,985,828; and IL-10R antibodies are described, e.g., in U.S. Pat. No. 5,863,796, all of which are incorporated herein by reference.

“PD-1” refers to programmed death receptor 1. “PD-L1” , also known as B7-H1 or B7-4, is one of the binding partners of PD-1. As used herein PD-1 and PD-L1 refer to human or mouse proteins. Human PD-L1 polypeptide sequence is provided in, e.g., GenBank Accession number AAF25807, and the mouse polypeptide sequence is provided, e.g., in GenBank Accession number AAG31810. Human PD-1 polypeptide sequence is provided in, e.g., GenBank Accession number NP_{—}005009; mouse PD-1 polyeptide sequence is provided, e.g., in GenBank Accession number AAI20603.

A “soluble receptor” as used herein is the extracellular domain of a receptor protein.

A “fusion protein” is a hybrid protein expressed by a nucleic acid molecule comprising nucleotide sequences of at least heterologous two genes encoding at least two heterologous proteins. For example, a fusion protein can comprise at least part of an IL-10R1 polypeptide, e.g., the extracellular domain, fused with an Fc region of an antibody.

A “pegylated” or “PEG” protein is a protein or polypeptide having one or more polyethylene glycol, molecules covalently attached to one or more than one amino acid residue of the IL-10 protein via a linker, such that the attachment is stable. The terms “monopegylated” and “mono-PEG”, mean that one polyethylene glycol molecule is covalently attached to a single amino acid residue on one subunit of a multimeric protein via a linker. The average molecular weight of the PEG moiety is preferably between about 5,000 and about 50,000 daltons. The method or site of PEG attachment to the protein or polypeptide is not critical, but preferably the pegylation does not alter, or only minimally alters, the activity of the biologically active molecule. Preferably, the increase in half-life is greater than any decrease in biological activity. For example pegylated IL-10R or PEG-IL-10R may comprise the extracellular domain of IL-10R1 covalently linked to at least one PEG molecule.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they retain, or are modified to comprise, a ligand-specific binding domain. The antibody herein is directed against an “antigen” of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or ligand such as a growth factor. Exemplary antigens include those polypeptides.

“Antibody fragments” comprise a portion, of a full length antibody, generally the antigen binding or variable region thereof. Examples of antibody fragments include Fab, Fab′, F(ab′)₂, and Fv fragments; single-chain antibody molecules; diabodies; linear antibodies; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal, antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., (1991) Nature 352:624-628 and Marks et al., (1991) J. Mol. Biol. 222:581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81:6851-6855.)

The term “hypervariable region” when used herein refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (HI), 53-55 (H2) and 96-101 (H3) in the heavy chain variable domain; Chothia and Lesk (1987) J. Mol. Biol. 196:901-917). “Framework” or “FR” residues are those variable domain residues other than the hypervariable region residues as herein defined.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and ail or substantially all of the FR regions are those of a human immunoglobulin sequence. The humanized antibody optionally also will comprise at least a portion of an immunoglobulin constant region (Fc), typically that of a human immunoglobulin. For further details, see Jones et al., (1986) Nature 321:522-525; Riechmann et al., (1988) Nature 332:323-329; and Presta (1992) Curr. Op. Struct. Biol. 2:593-596. In one embodiment humanized IL-10 antibodies as described in US 2005/0101770

### II. General.

The present invention provides methods of treating chronic or persistent viral, infections with a combination of a vaccine and an antagonist against an immunosuppressive cytokine.

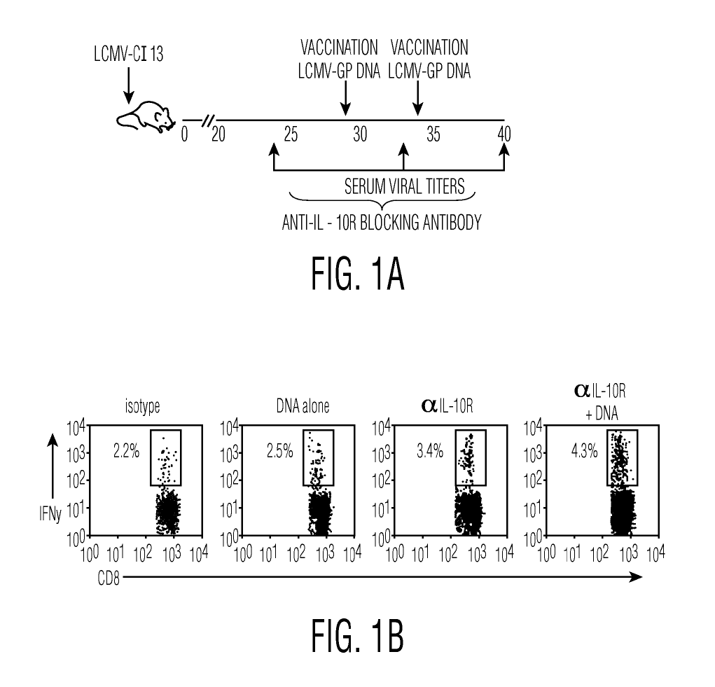

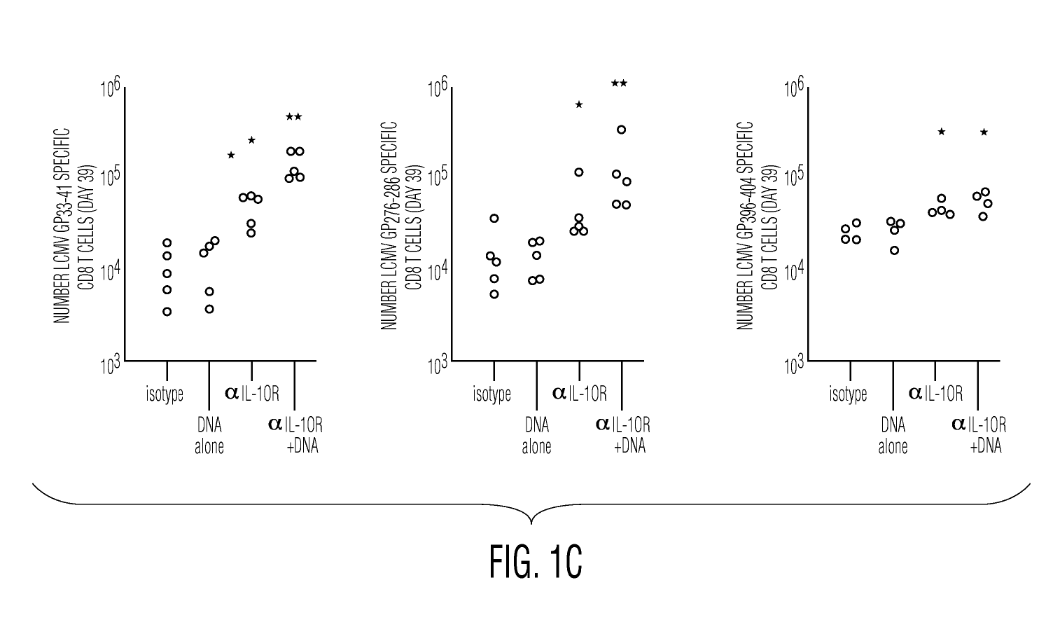

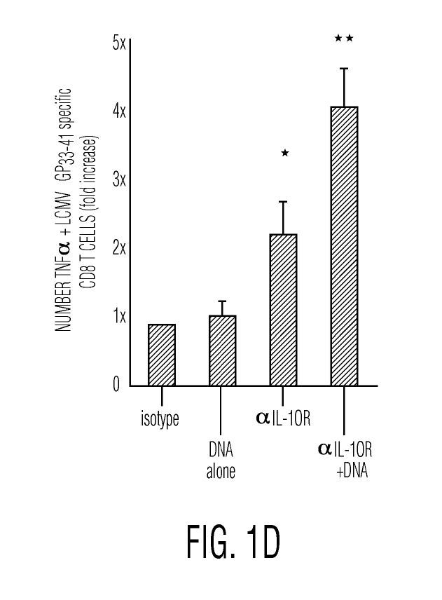

To determine how the immunosuppressive environment affects T cell responsiveness to vaccination during persistent viral infection C57BL/6 mice were infected with lymphocytic choriomeningitis virus Clone 13 (LCMV CI 13), as described below. Infection with LCMV-CI 13 rapidly induces high level expression of IL-10 that suppresses antiviral immunity and leads to viral persistence (see, e.g., Brooks, et al. (2006) supra; Ejmaes, et al. (2006) supra; and Ahmed, et al. (1984) J. Exp, Med. 160:521-540). To determine whether IL-10 inhibits responsiveness to therapeutic vaccination LCMV-CI 13 persistently infected mice were treated with 1) isotype control antibody alone; 2) treated with isotype control antibody and vaccinated with a DNA plasmid encoding the entire glycoprotein (GP) sequence of LCMV; 3) treated with anti-IL-10R blocking antibody; or 4) treated with a combination of anti-IL-10R blocking antibody plus DNA vaccination. Antibody treatment was initiated on day 25 after CI 13 infection and administered every 3 days for 5-6 treatments. DNA vaccination was administered on day 29 and day 34 following virus infection (i.e., 4 and 9 days after the initiation of anti-IL-10R therapy). The treatment regimen is illustrated in FIG. 1a.

Consistent with the inability of therapeutic vaccination to stimulate T cell responses during persistent infection (see, e.g., Wherry et al. (2005), J. Virol. 79:8960-8968), DNA vaccination alone had no effect on the frequency or number of virus-specific CD8 T cells compared to untreated animals (FIG. 1b and c). On the other hand, IL-10R blockade alone increased the frequency and number of IFNγ-producing CD8 T cells against multiple LCMV epitopes (FIG. 1b and c), indicating that IL-10 suppresses T cell activity throughout persistent infection and that blocking IL-10 activity alone can boost T cell immunity. Importantly, IL-10R blockade combined with DNA vaccination dramatically enhanced T cell responses compared with either DNA vaccine or anti-IL-10R therapy alone (FIG. 1c). Anti-IL-10R plus DNA vaccine dual therapy induced a 2-fold increase in the frequency and greater than 5-fold increase in the number of IFNγ-producing, virus-specific CD8 T cells. A similar enhancement of CD8 T cell immunity was observed by MHC class I tetramer staining. Although IL-10R blockade alone increased the number of LCMV nucleoprotein (NP)₃₉₄₋₄₀₄-specific CD8 T cells compared to isotype treatment, DNA vaccine (which contained only LCMV-GP epitopes and not LCMV-NP epitopes) plus anti-IL-10R therapy did not enhance LCMV-NP₃₉₅₋₄₀₄ specific CD8 T cell responses, demonstrating that T cell stimulation was due to the DNA vaccine and not a secondary effect of heightened immune activation (FIG. 1c). Vaccination with the parental control DNA plasmid that did not encode LCMV-GP failed to further enhance T cell, responses when combined with IL-10R blockade, indicating that the elevated T cell activity did not result from the introduction of exogenous DNA.

IL-10R blockade followed by vaccination significantly increased the number of functional virus-specific CD8 T cells (FIG. 1d). Whereas vaccination alone did not enhance virus-specific CD8 T cell function, and IL-10R blockade alone only increased the number of TNFα producing virus-specific T cells approximately 2-fold, IL-10R blockade combined with vaccination stimulated a 4-fold increase in the number of functional CD8 T cells that produced TNFα (FIG. 1d). The treatments did not substantially increase the frequency of TNFα producing CD8 T cells. Rather, only an increase in the absolute number of functional, cytokine producing CD8 T cells was observed (FIG. 1d).

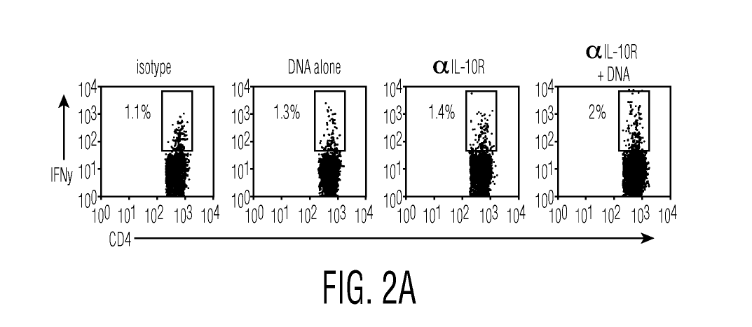

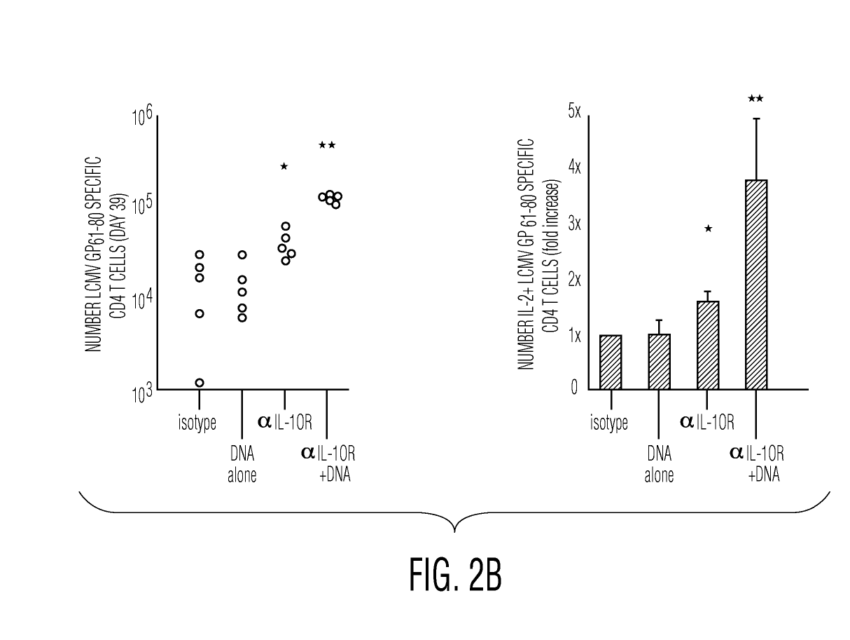

IL-10R blockade alone had a less dramatic effect on CD4 T cells than that observed for CD8 T cells, inducing a small but significant (p<0.05) 1.5 to 2-fold increase in the number of IFNγ-producing CD4 T cells (FIG. 2a and b). On the other hand, IL-10R blockade plus DNA vaccine therapy substantially increased the frequency and particularly the number (a 6-fold increase compared to isotype treatment) of IFNγ-producing CD4 T cells (FIG. 2a and b). A similar increase in the number of LCMV-GP₆₁₋₈₀-specific CD4 T cells was observed using MUG class II tetramers (data not shown). Further, while either DNA vaccine or anti-IL-10R therapy alone only modestly affected the number of IL-2 producing CD4 T cells, co-treatment stimulated a 4-fold increase in the number of these functional cells (FIG. 2c). These data demonstrate that an otherwise ineffective vaccination can stimulate robust T cell responses during persistent viral infection if IL-10 mediated immunosuppression is neutralized.

To address whether the increased T cell function was due to reactivation of previously exhausted T cells or from de novo priming of naive T cell precursors, Thy1.1+ T cell receptor (TcR) transgenic (tg) CD4 T cells (SMARTA cells; specific to LCMV-GP₆₁₋₈₀ peptide) and Thy1.1+ TcR transgenic CD8 T cells (P14 cells; specific to LCMV GP₃₃₋₄₁ peptide) were co-transferred into Thy1.2+ C57BL/8 mice and the mice were subsequently infected with LCMV-CI 13. The co-transfer enabled analysts of LCMV-specific CD4 and CD8 T cells present from the beginning of infection and that could be distinguished from endogenous (i.e., host-derived) antiviral T cells based on Thy1.1 versus Thy1.2 expression. Physiologic numbers of these tg T cells were transferred to ensure that they respond similarly to their endogenous CD4 (FIG. 2) and CDS (FIG. 1) T cell counterparts (see. e.g., Brooks, et al. (2006). J Clin Invest 116:1675-1685).

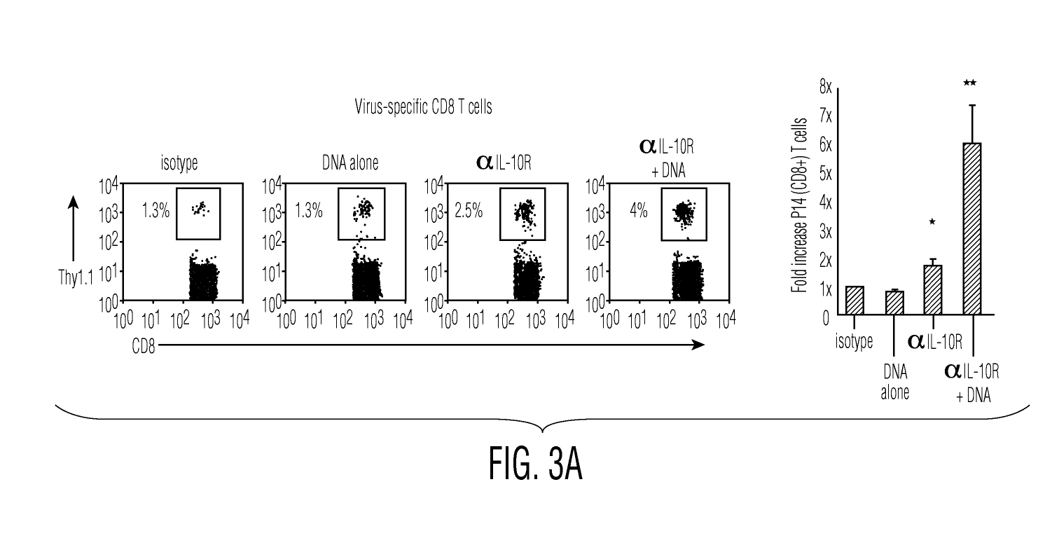

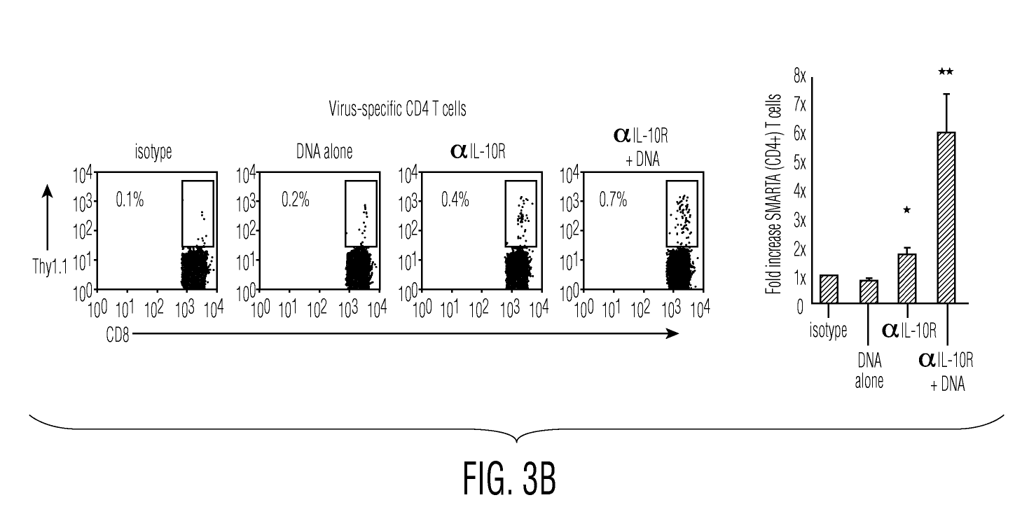

Similar to endogenous T cell responses (FIGS. 1 and 2) DMA vaccine alone did not increase the number of virus-specific tg CD8 or CD4 T cells, whereas IL-10R blockade stimulated a 2- and 4-fold increase in the number of tg CD8 and CD4 T cells, respectively (FIG. 3). IL-10R blockade in combination with DNA vaccination induced a dramatic 6-fold increase in the number of tg virus-specific CD8 T cells compared to isotype control or DNA vaccine alone treated mice and a ˜3-fold increase when compared to IL-10R blockade alone (FIG. 3a).

Similarly, IL-10R blockade in combination with vaccination stimulated an 11-fold increase in the number of virus-specific tg CD4 T cells compared to isotype control or vaccine alone and a 4-fold increase versus IL-10R blockade therapy alone (FIG. 3b). Like their endogenous counterparts, the combination therapy also elevated the number of functional virus-specific T cells (i.e., TNFα producing tg CD8 T cells and IL-2 producing tg CD4 T cells). Thus, IL-10R blockade permits the previously exhausted T cells to respond to vaccination.

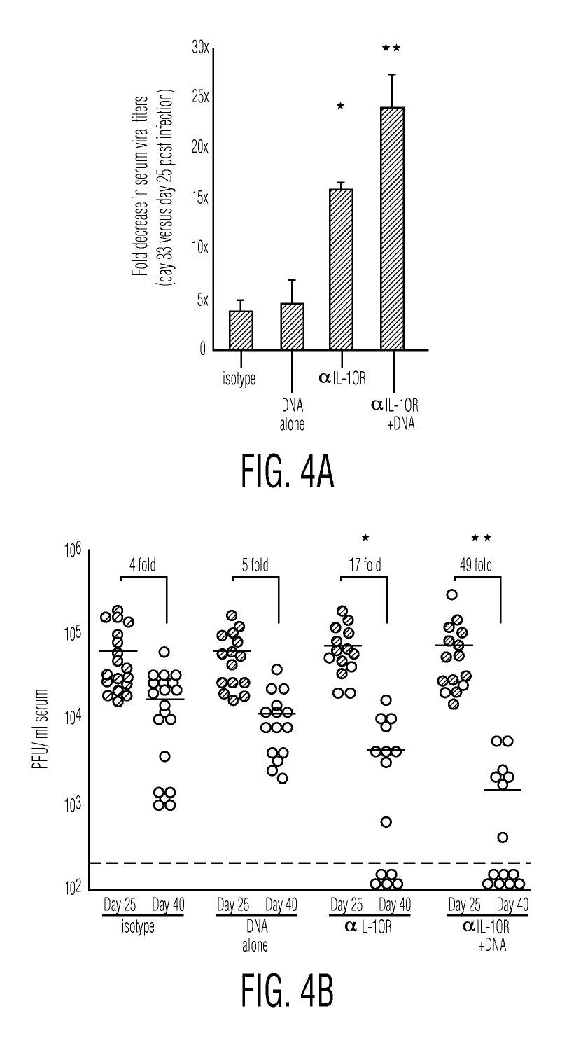

To determine if the increased number of functional virus-specific T cells following IL-10R blockade and vaccination enhanced control of infection, virus liters were quantified following treatment (i.e., day 33 after infection and; therefore, following 3 anti-IL-10R antibody treatments and/or a single DNA vaccination) and compared to pre-treatment levels. Consistent with the inability to stimulate T cell responses. DNA vaccination alone had no impact on the control of viral replication (FIG. 4a), In contrast, IL-10R blockade induced a 15-fold decrease in viral titers (FIG. 4a).

IL-10R blockade in combination with vaccination induced a 24-fold decrease in virus titers, indicating that the enhanced T cell responses arising from combination therapy were better equipped to subdue viral, replication than IL-10R blockade alone, it should be noted that the accelerated viral clearance following co-treatment was observed after only a single DNA vaccination, implying that T cells rapidly become responsive when the immunosuppressive signals are neutralized. Following treatment, viral titers were similar in isotype and DNA vaccinated mice through 40 days after infection; whereas, viral replication was significantly (p<0.05) decreased in anti-IL-10R treated mice, foiling 17-fold from the initiation to the completion of therapy (FIG. 4b). Importantly, the initial accelerated control of virus replication in anti-IL-10R and DNA vaccine co-treated mice resulted in an enhanced ability to control and eliminate persistent viral replication, ultimately resulting in a 49-fold decrease in virus replication following the resolution of therapy (FIG. 4b).

There was a discrepancy in the ability of some animals to eliminate virus infection following IL-10R blockade alone or in combination with DNA vaccination (FIG. 4b). Whereas some animals completely cleared virus infection following therapy, other similarly treated animals still retained virus replication. It should be noted that FIG. 4b illustrates the combined data from multiple experiments and that pre-therapy virus titers varied between experiments. However, generally animals with higher pre-treatment virus titers corresponded to higher levels of virus replication after treatment and results were significant within experiments. The difference in virus clearance kinetics following treatment was not due to the gender or the age of the mice. Importantly, viral replication remained absent following IL-10R blockade (>150 days post infection) and treated mice were protected from subsequent re-infection (data not shown). Interestingly, anti-IL-10R antibody treatment every 3 days boosted T cell responses leading to control of persistent viral infection, whereas weekly treatments, including treatment with twice the required dose of antibody, were ineffective. Thus, the timing of antibody therapy is an important determinant for therapeutic efficacy. IL-10R blockade in combination with the control DNA vaccine (i.e., not encoding LCMV GP) did not affect virus liters compared to anti-IL-10R treatment alone, again demonstrating that the enhanced effect of DNA vaccine was due to stimulation of LCMV-specific cells. Thus, neutralizing IL-10 mediated immunosuppression facilitates the induction of robust virus-specific T cell, responses with an enhanced ability to control persistent viral infection.

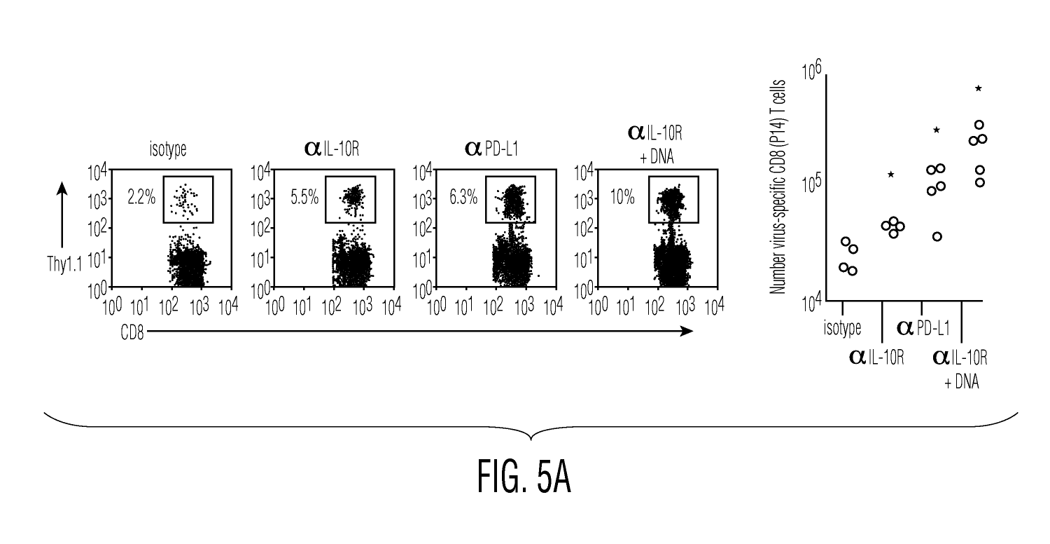

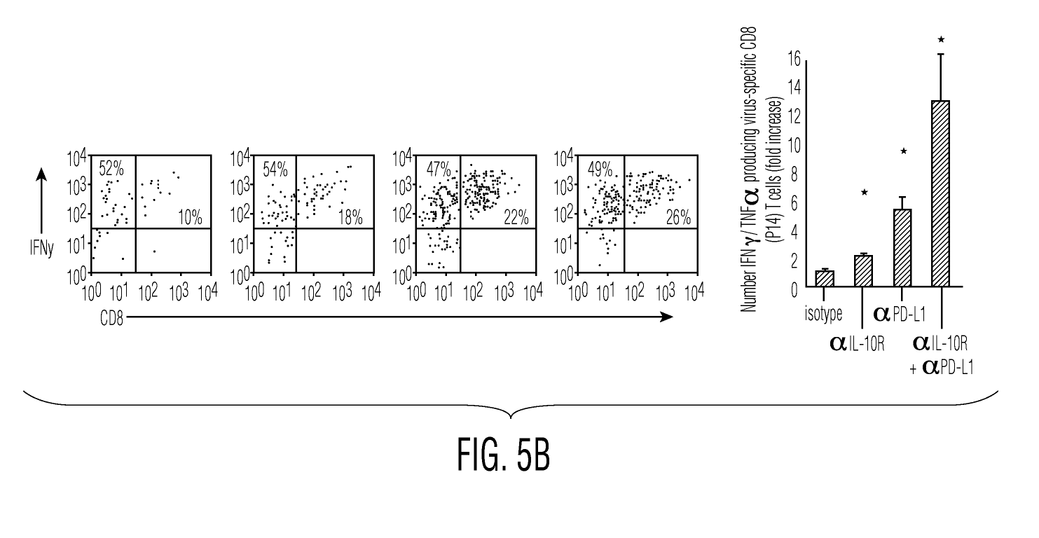

Combination therapies that antagonize virally-mediated immunosuppressive pathways showed restoration and boosting of anti-viral T cell activity, as well as synergism when compared to single agents. One such combination therapy was the use of antibodies against IL-10R and PD-L1 to eliminate persistent viral infection in the LCMV mouse model described above. Administration of blocking antibodies against PD-L1 and IL-10 in the mouse model increased the level of previously exhausted virus-specific T cells (see FIG. 5a), and also increased the level of INFγ/TNFα producing cells (see FIG. 5b).

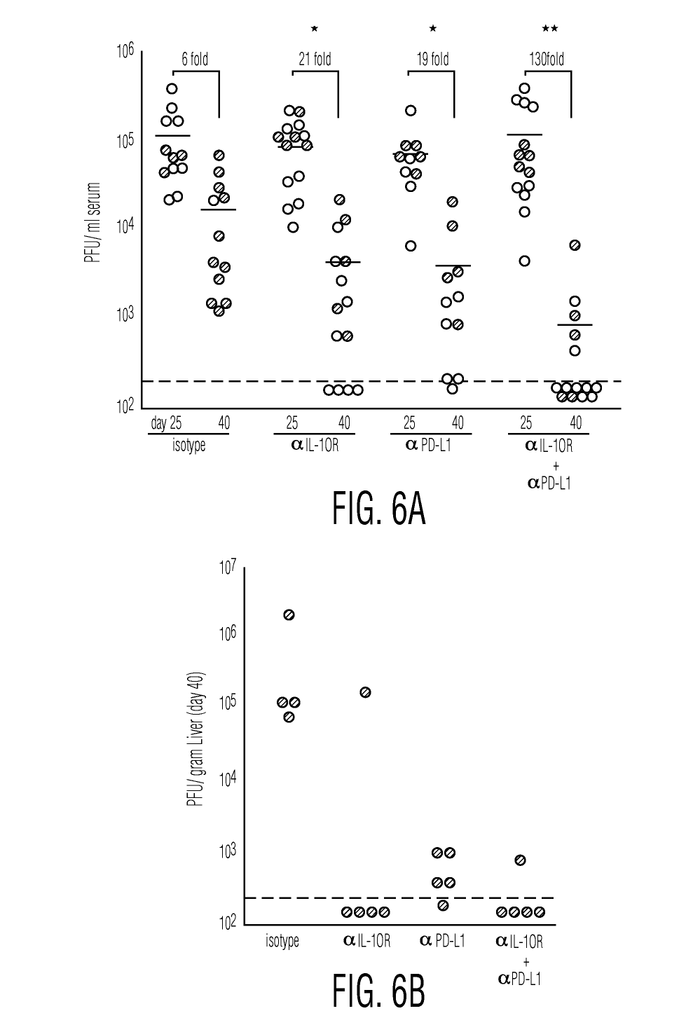

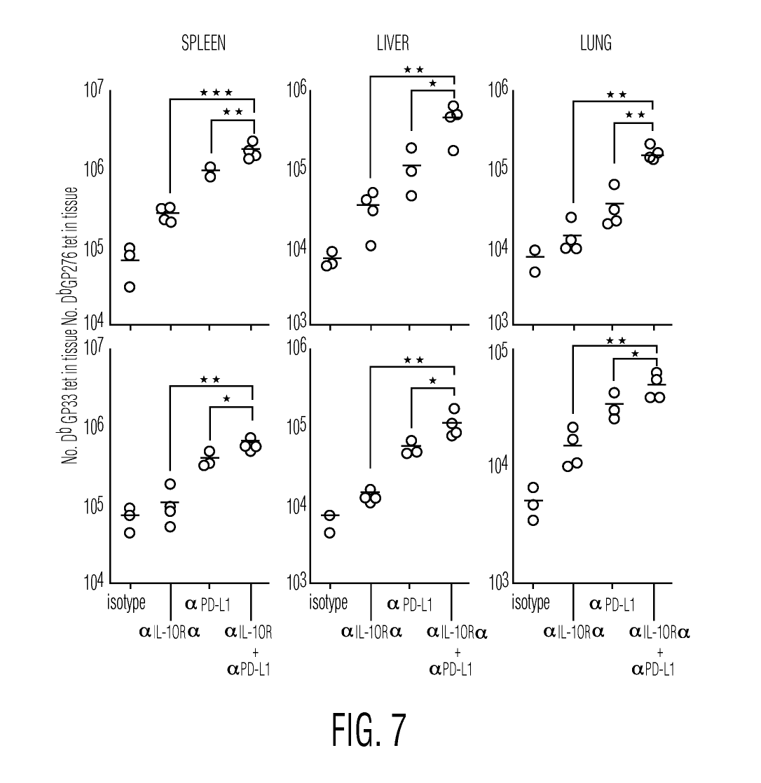

It was also determined that dual IL-10R/PD-L1 blockade also led to an enhanced ability to control persistent virus replication. Following the two week therapeutic regimen of administering IL-10R and PD-L1 antibodies, alone or in combination, mice receiving the combination therapy showed a significant increase viral clearance as compared to single agent therapy and control animals (see, e.g., FIG. 6a). Viral titers were also reduced in the livers of antibody treated animals compared to the isotype control treated animals (see FIG. 6b). The combination of IL-10 and PD-L1 antagonism appears to induce the recovery of exhausted CD8+ T cells more effectively than each treatment alone (see. e.g., FIG. 7).

### III. Expression Vectors

A wide variety of host/expression vector combinations (i.e., expression systems) may be employed in expressing the immunogenic polypeptides of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, SV40 and pMal-C2, pET, pGEX (Smith, et al., Gene 67:31-40, 1988), pMB9 and their derivatives, plasmids such as RP4; gram positive vectors such as Strep, gardonii; phage DNAS, e.g., the numerous derivatives of phage I, e.g., NM989, and other phage DNA, e.g., M13 and filamentous single stranded phage DNA; yeast plasmids such as the 2 m plasmid or derivatives thereof: vectors useful in eukaryotic cells, such as vectors useful in insect or mammalian cells; vectors derived from combinations of plasmids and phage DNAs, such as plasmids that have been modified to employ phage DNA or other expression control sequences; and the like.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region. (Benoist and Chambon (1983.) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. U.S.A. 78:1441-1445), the regulatory sequences of the metallothionein gene (Brinster, et al., (1982) Nature 296:39-42): prokaryotic expression vectors such as the b-lactamase promoter (Vilta-Komaroff, et al., (1978) Proc. Natl. Acad. Sci. U.S.A. 75:3727-3731), or the tac promoter (DeBoer, et al., (1983) Proc. Natl. Acad. Sci. U.S.A. 80:21-25); see also “Useful proteins from recombinant bacteria” in Scientific American. 242:74-94, 1980; promoter elements from, yeast or other fungi such as the Gal 4 promoter, the ADC (alcohol dehydrogenase) promoter, PGK (phosphogiycerol kinase) promoter, alkaline phosphatase promoter; and control regions that exhibit hematopoietic tissue specificity, in particular: immunoglobin gene control region, which is active in lymphoid cells (Grosschedl et al., (1984) Cell 38:647; Adames et al., (1985) Nature 318:533; Alexander et al., (1987) Mol. Cell Biol. 7:1436); beta-globin gene control region which is active in myeloid cells (Mogram, et al., (1985) Nature 31.5:338-340; Kollias, et al., (1986) Cell 46:89-94), hematopoietic stem cell differentiation factor promoters; erythropoietin receptor promoter (Maouche, et al., (1991) Blood 15:2557), etc; and control regions that exhibit mucosal, epithelial cell specificity.

Preferred vectors, particularly for cellular assays in vitro and vaccination in vivo or ex vivo, are viral vectors, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses with desirable cellular tropism. In a specific embodiment, a vaccinia virus vector is used to infect dendritic cells. In another specific embodiment, a baculovirus vector that expresses EBNA-1 is prepared. Thus, a vector encoding an immunogenic polypeptide can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and vaccination procedures are DNA-based vectors and retroviral vectors. Methods for constructing and using viral vectors are known in the art (see, e.g., Miller and Rosman (1992) Bio Techniques 7:980-990). Preferably, the viral vectors are replication defective, that is, they are unable to replicate autonomously in the target cell. Preferably, the replication defective virus is a minimal virus, i.e., it retains only the sequences of its genome which are necessary for encapsidating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), vaccinia virus, and the like. Examples of particular vectors include, but are not limited to, a defective herpes virus 1 (HSV1) vector (Kaplitt et al., (1991) Molec. Cell. Neurosci. 2:320-330: International Patent Publication No. WO 94/21807, published Sep. 29, 1994; International Patent Publication No. WO 92/05263, published Apr. 2, 1994): an attenuated adenovirus vector, such as the vector described by Stratford-Perricaudet, et al. (1992) J. Clin. Invest. 90:626-630; see also La Salle, et al., (1993) Science 259:988-990); and a defective adeno-associated virus vector (Samufski, et al. (1987) J. Virol. 61:3096-3101; Samufski, et al., (1989) J. Virol. 63:3822-3828; Lebkowski, et al. (1988) Mol. Cell. Biol. 8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see WO94/26914). Those adenoviruses of animal origin which can be used within the scope of the present invention include adenoviruses of canine, bovine, murine (example: Mav1, Beard, et al. (1990) Virology 175(l):81-90), ovine, porcine, avian, and simian (example: SAV) origin. Preferably, the adenovirus of animal origin is a canine adenovirus, more preferably a CAV2 adenovirus (e.g. Manhattan or A26/61 strain (ATCC VR-800), for example). Various replication defective adenovirus and minimum adenovirus vectors have been described (WO94/26914, WO95/02697, WO94/28938, WO94/28152, WO94/12649, WO95/02697 WO96/22378). The replication defective recombinant adenoviruses according to the invention can be prepared by any technique known to the person skilled in the art (Levrero, et al. (1991) Gene 101:195; EP 185 573; Graham (1984) EMBO J. 3:2917: Graham, et al. (1977) J. Gen. Virol. 36:59). Recombinant adenovirus is an efficient and non-perturbing vector for human dendritic ceils (Zhong et al. (1999) Eur. J. Immunol. 29(3):964-72: DiNicola et al. (1998) Cancer Gene Ther. 5:350-6, 1998). Recombinant adenoviruses are recovered and purified using standard molecular biological techniques, which are well known to one of ordinary skill in the art.

Adeno-associated viruses. The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see WO 91/18088; WO 93/09239; U.S. Pat. No. 4,797,368, U.S. Pat. No. 5,139,941, EP 488 528). The replication, defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques. These viral vectors are also effective for gene transfer into human dendritic cells (DiNicola et al., supra).

Retrovirus vectors. In another embodiment the gene can be introduced in a retroviral vector, e.g., as described in Anderson, et al., U.S. Pat. No. 5,399,346; Mann, et al., (1983) Cell 33:153; Temin, et al., U.S. Pat. No. 4,650,764; Temin, et al., U.S. Pat. No. 4,980,289; Markowitz, et al. (1998) J. Virol. 62:1120; Temin, et al., U.S. Pat. No. 5,124,263; EP 453242, EP178220; International Patent Publication No. WO 95/07358, published Mar. 16, 1995, by Dougherty, et al.; and Kuo, et al. (1993) Blood 82:845. The retroviruses are integrating viruses which infect dividing cells. The retrovirus genome includes two LTRs, an encapsidation sequence and three coding regions (gag, pot and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PAS 17 (U.S. Pat. No. 4,861,719); the PsiCRIP cell line (WO 90/02806) and the GP+envAm-12 eel! line (WO 89/07150). In addition, the recombinant retroviral vectors can contain modifications within the LTRs for suppressing transcriptional activity as well as extensive encapsidation sequences which may include a part of the gag gene (Bender, et al. (1987) J. Virol. 61:1639). Recombinant retroviral vectors are purified by standard techniques known to those having ordinary skill in the art.

Retrovirus vectors can also be introduced by DNA viruses, which permits one cycle of retroviral replication and amplifies tranfection efficiency (see WO 95/22617, WO 95/26411, WO 96/39036, WO 97/19182).

Lentivirus vectors. In another embodiment, lentiviral vectors are can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini (1998) Curr. Opin. Biotechnol. 9:457-63; see also Zufferey, et al. (1998) J. Virol. 72:9873-80). Lentiviral packaging cell, lines are available and known generally in the art. They facilitate the production of high-tiler lentivirus vectors for gene therapy. An example is a tetracycline-inducible VSV-G pseudotyped lentivirus packaging cell line which can generate virus particles at titers greater than 106 IU/ml for at least 3 to 4 days (Kafri, et al., (1999) J. Virol. 73: 576-584). The vector produced by the inducible cell line can be concentrated as needed for efficiently transducing nondividing cells in vitro and in vivo.

Vaccinia virus vectors. Vaccinia virus is a member of the pox virus family and is characterized by its large size and complexity. Vaccinia virus DNA is double-stranded and terminally crosslinked so that a single stranded circle is formed upon denaturation of the DNA. The virus has been used for approximately 200 years in a vaccine against smallpox and the properties of the virus when used in a vaccine are known (Paoletti (1996) Proc. Natl. Acad. Sci. U.S.A. 93:11.349-53; and Ellner (1998) Infection 26:263-9). The risks of vaccination with vaccinia virus are well known and well defined and the virus is considered relatively benign. Vaccinia virus vectors can be used for the insertion and expression of foreign genes. The basic technique of inserting foreign genes into the vaccinia vector and creating synthetic recombinants of the vaccinia virus has been described (see U.S. Pat. No, 4.603,112. U.S. Pat No. 4,722,848. U.S. Pat. No. 4,769,330 and U.S. Pat. No. 5.364,773). A large number of foreign (i.e. non-vaccinia) genes have been expressed in vaccinia, often resulting in protective immunity (reviewed by Yamanouchi, Barrett, and Kai, (1998) Rev. Sci. Tech. 17:641-53; Yokoyama, et al., (1997) J. Vet. Med Sci. 59:311-22; and see Osterhaus, et al. (1998) Vaccine 16:1479-81: and Gherardi et al., (1999) J. Immunol 162:6724-33). Vaccinia virus may be inappropriate for administration to immunocompromised or immunosuppressed individuals. Alternative pox viruses which may be used in the invention include Fowl pox, AV-pox, and modified vaccinia Ankara (MVA) virus.

Nonviral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Feigner, et. al. (1987) Proc. Natl. Acad. Sci. U.S.A. 84:7413-7417; Felgner and Ringold (1989) Science 337:387-388; see Mackey, et al. (1988) Proc. Natl. Acad. Sci. U.S.A. 85:8027-8031; Ulmer, et al. (1993) Science 259:1745-1748). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO 95/21931).

Alternatively, non-viral DNA vectors for gene therapy can be introduced into the desired host cells by methods known, in the art, e.g., electroporation, microinjection, cell fusion. DEAE dextran, calcium phosphate precipitation, use of a gene gun (ballistic transfection; see, e.g., U.S. Pat. No. 5,204,253, U.S. Pat. No. 5,853,663, U.S. Pat. No. 5,885,795, and U.S. Pat. No. 5,702,384 and see Sanford, TIB-TECH, 6:299-302, 1988: Fynan et al., (1993) Proc. Natl. Acad. Sci. U.S.A. 90:11478-11482; and Yang et al., (1990) Proc. Natl. Acad. Sci. U.S.A. 87:1568-9572), or use of a DNA vector transporter (see, e.g., Wu, et al., (1992) J. Biol. Chem. 267:963-967; Wu and Wu (1988) J. Biol. Chem. 263:14621-14624; Hartmut, et al., Canadian Patent Application No. 2,012,311, filed Mar. 15, 1990; Williams, et al., (1991) Proc. Natl. Acad. Sci. USA 88:2726-2730). Receptor- mediated DNA delivery approaches can also be used (Curiel, et al., (1992) Hum. Gene Ther. 3; 147-154; Wu and Wu, (1987) J. Biol. Chem. 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir, et al., (1998) C.P. Acad. Sci. 321:893; WO 99/01157; WO 99/01158: WO 99/01175).

### IV. IL-10 or IL-10R Antagonists

Antagonists of IL-10 are administered in conjunction with standard vaccine therapies. Antagonists of IL-10 as used herein encompass neutralizing antibodies or fragments thereof IL-1.0 antisense DNA, IL-10R soluble receptor and/or receptor fusion proteins (e.g., Fc fusion proteins), IL-10 mutant proteins that bind to IL-10R, but do not cause signaling of the receptor complex. In one particular embodiment, IL-10R fusion proteins are contemplated.

### V. Antibodies

The preferred protein to be purified according to the present invention is an antibody. The antibody herein, is directed against an antigen of interest. Preferably, the antigen is a biologically important polypeptide and administration of the antibody to a mammal suffering from a disease or disorder can result in a therapeutic benefit in that mammal. Where the antigen is a polypeptide, it may be a transmembrane molecule (e.g., cytokine receptor) or ligand such as a growth factor or cytokine.

The antibody herein is directed against an antigen of interest, e.g., human IL-10. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. cytokine receptor) or ligand such as a growth factor or cytokine.

Polyclonal antibodies are preferably raised in animals by multiple subcutaneous (sc) or intraperitoneal, (ip) injections of the relevant antigen and an adjuvant. It may be useful to conjugate the antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride. SOCl₂, or R¹N=C=NR, where R and R¹ are different alkyl groups.

Animals are immunized against the antigen, immunogenic conjugates, or derivatives by combining, e.g., 100 μg or 5 μg of the protein or conjugate (for rabbits or mice. respectively) with 3 volumes of Freund's complete adjuvant and injecting the solution intradermally at multiple sites. One month later the animals are boosted with ⅕ to 1/10 the original, amount of antigen or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., (1975) Nature, 256:495, or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk institute Cell Distribution Center, San Diego, Calif. USA, and SP-2 or X63-Ag8-653 cells available from, the American Type Culture Collection, Rockville, Md. USA. Human myeloma and mouse-human heteromyeloma cell lines also have been described for the production of human monoclonal antibodies (Kozbor (1984) J. Immunol. 133:3001; Brodeur et al., Monoclonal Antibody Production Techniques and Applications, pp. 51 -63 (Marcel Dekker, Inc., New York, 1987)).

Culture medium in which hybridoma cells are growing is assayed for production of monoclonal antibodies directed against, the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Coding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, Protein A-Sepharose, hydroxyapatite chromatography, gel electrophoresis, dialysis, or affinity chromatography. Preferably the Protein A chromatography procedure described herein is used.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA, Once isolated, the DNA may be placed into expression vectors, which are then transfected into host cells such as E. coli cells, simian COS ceils, Chinese hamster ovary (CHO) cells, or myeloma cells that do not otherwise produce immunoglobulin protein, to obtain the synthesis of monoclonal antibodies in the recombinant host cells.

The DNA also may be modified, for example, by substituting the coding sequence for human heavy-arid light-chain constant domains in place of the homologous murine sequences (U.S. Pat, No, 4,816,567: Morrison, et al. (1984) Proc. Natl. Acad. Sci. USA, 81:6851), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent, antibody comprising one antigen-combining site having specificity for an antigen and another antigen-combining site having specificity for a different antigen.

In a further embodiment, monoclonal, antibodies can be isolated from antibody phage libraries generated using the techniques described in MeCafferty et al., (1990) Nature, 348:552-554. Clackson et al., (1991) Nature, 352:624-628 and Marks et al. (1991) J. Mol. Biol., 222:581-597 describe the isolation of murine and human antibodies, respectively, using phage libraries. Subsequent publications describe the production of high affinity (nM range) human antibodies by chain shuffling (Marks et al. (1992) Bio/Technology, 10:779-783), as well as combinatorial infection and in vivo recombination as a strategy for constructing very large phage libraries (Waterhouse et al., (1993) Nuc. Acids. Res., 21:2265-2266). Thus, these techniques are viable alternatives to traditional hybridoma techniques for isolation of monoclonal antibodies.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., (1986) Nature, 321:522-525; Riechmann et al., (1988) Nature, 332:323-327; Verhoeyen et al., (1988) Science, 239:1534-1536), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (Sims et al., (1993) J. Immunol., 151:2296). Another method uses a particular framework derived from the consensus sequence of all human antibodies of a particular subgroup of light or heavy chains. The same framework may be used for several different humanized antibodies (Carter et al., (1992) Proc. Natl. Acad. Sci. USA, 89:4285; Presta et al. (1993) J. Immunol., 151:2623).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region, (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., (1993) Proc. Nat. Acad. Sci. USA, 90:2551; Jakobovits et al., (1993) Nature, 362:255-258; Bruggermann et al., (1993) Year in Immuno., 7:33; and Duchosal et al. (1992) Nature 355:258. Human antibodies can also be derived from phage-display libraries (Hoogenboom et al., (1991) J. Mol. Biol., 227:381; Marks et al., (1991) J. Mol. Biol., 222:581-597: Vaughan et al. (1996) Nature Biotech 14:309).

Various techniques have been developed for trip production of antibody fragments. Traditionally, these fragments were derived via proteolytic digestion of intact antibodies (see, e.g., Morimoto et al. (1992) Journal of Biochemical and Biophysical Methods 24:1.07-1.17 and Brennan et al. (1985) Science, 229:81). However, these fragments can now be produced directly by recombinant host cells. For example, the antibody fragments can be isolated from the antibody phage libraries discussed above. Alternatively. Fab′-SH fragments can be directly recovered from E. coli and chemically coupled to form F(ab′)₂ fragments (Carter et al., (1992) Bio/Technology 10:163-167). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., (1983) Nature, 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., (1991) EMBO J., 10:3655-3659.

According to another approach described in WO96/2701!, the interface between a pair of antibody molecules can be engineered to maximize the percentage of heterodimers which are recovered from recombinant cell culture. The preferred interface comprises at least a pan of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., (1985) Science, 229:81 describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., (1992) J. Exp. Med., 175: 217-225 describe the production, of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., (1992) J. Immunol, 148(5): 1547-1.553. The leucine zipper peptides from the Fos and Jun proteins were linked. to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., (1993) Proc. Natl. Acad. Sci. USA. 90:6444-6448 has provided an alternative mechanism for making bispecific antibody fragments. The fragments comprise a heavy-chain variable domain (V_{H}) connected to a light-chain variable domain V(_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain. Fv (sFv) dimers has also been reported. See Gruber et al., (1994) J. Immunol., 152:5368. Alternatively, the antibodies can be “linear antibodies” as described in Zapata et al. (1995) Protein Eng. 8(10):1057-1.062. Briefly, these antibodies comprise a pair of tandem Fd segments (V_{H}-C_{H} 1-V_{H}-C_{H}1) which form a pair of antigen binding regions. Linear antibodies can be bispecific or monospecific. Dual variable domain antibodies, as described in US 2007/0071675, are also contemplated.

In one embodiment, antagonists antibodies of the present invention are humanized anti-IL-10 antibodies as described in, e.g., US 2005/0101770 and US 2007/0178097, both of which are hereby incorporated by reference.

### VI. Vaccination and Immunotherapy Strategies

Various strategies can be employed to vaccinate subjects against chronic or persistent viral infections. The polypeptide vaccine formulations can be delivered by subcutaneous (s.c.), intraperitoneal (i.p.), intramuscular (i.m.), subdermal (s.d.), intradermal (i.d.), or by administration to antigen presenting cells ex vivo followed by administration of the cells to the subject. Prior to administration to the subject, the antigen presenting cells may be induced to mature.

Similarly, any of the gene delivery methods described above can be used to administer a vector vaccine to a subject, such as naked DNA and RNA delivery, e.g., by gene gun or direct injection. (

Vaccination effectiveness may be enhanced by co-administration of an immunostimulatory molecule (Salgaller and Lodge (1998) J. Surg. Oncol. 68:122), such as an immunostimulatory, immunopotentiating, or pro-inflammatory cytokine, lymphokine, or chemokine with the vaccine, particularly with a vector vaccine. For example, cytokines or cytokine genes such as interleukin (IL)-1, IL-2, IL-3, IL-4, IL-12, IL-13, granulocyte-macrophage (GM)-colony stimulating factor (CSF) and other colony stimulating factors, macrophage inflammatory factor, Flt3 ligand (Lyman (1998) Curr. Opin. Hematol. 5:192), as well as some key costimulatory molecules or their genes (e.g., B7.1, B7.2) can be used. These immunostimulatory molecules can be delivered systemically or locally as proteins or by expression of a vector that codes for expression of the molecule. Alternatively, as described herein, the vaccine can be administered with an antagonist of an immunosuppressive molecule, e.g., IL-10 or IL-10R. The techniques described above for delivery of the immunogenic polypeptide can also be employed for the immunostimulatory molecules.

Vaccination, and particularly immunotherapy may be accomplished through the targeting of dendritic cells (Steinman. (1996) J. Lab. Clin. Med. 128:531; Steinman, (1996) Exp. Hematol. 24:859; Taite et al., (1999) Leukemia 13:653; Avigan, (1999) Blood Rev. 13:51; DiNicola et al. (1998) Cytokines Cell. Mol. Ther. 4:265). Dendritic cells play a crucial role in the activation of T-cell dependent immunity. Proliferating dendritic cells can be used to capture protein antigens in an immunogenic form in situ and then present these antigens in a form that can be recognized by and stimulates T cells (see, e.g., Steiman (1996) Exper. Hematol. 24:859-862; Inaba, et al., (1998) J. Exp. Med. 188:2163-73 and U.S. Pat. No. 5.851.756). For ex vivo stimulation, dendritic cells are plated in culture dishes and exposed to (pulsed with) antigen in a sufficient amount and for a sufficient period of time to allow the antigen to bind to the dendritic cells. Additionally, dendritic cells may be transfected with DNA using a variety of physical or chemical as described by Zhong et al., (1999) Eur. J. Immunol. 29:964-72; Van Tendeloo, et al., (1998) Gene Ther. 5:700-7; Diebold et al., (1999) Hum. Gene Ther. 10:775-86; Francotte and Urbain (1985) Proc. Natl. Acad. Sci. USA 82:8149 and U.S. Pat. No. 5,891,432 (Casares et al. (1997) J. Exp. Med. 186:1.481-6). The pulsed cells can then be transplanted back to the subject undergoing treatment, e.g., by intravenous injection. Preferably autologous dendritic cells, i.e., dendritic cells obtained from the subject undergoing treatment, are used, although it may be possible to use MHC-Class II-matched dendritic cells, which may be obtained from a type-matched donor or by genetic engineering of dendritic cells to express the desired MHC molecules (and preferably suppress expression of undesirable MHC molecules.)

Preferably, the dendritic cells are specifically targeted in vivo for expression of viral peptides associated with viruses causing chronic or persistent infection. Various strategies are available for targeting dendritic cells in vivo by taking advantage of receptors that mediate antigen presentation, such as DEC-205 (Swiggard et al. (1995) Cell. Immunol. 165:302-11: Steinman (1996) Exp. Hematol. 24:859) and Fc receptors. Targeted viral vectors, discussed above, can also be used. Additionally, dendritic cells may be induced to mature in vitro after infection by the viral vector, prior to transplantation in vivo.

Mucosal vaccine strategies are particularly effective for many pathogenic viruses, since infection often occurs via the mucosa. Additionally, mucosal delivery of recombinant vaccinia virus vaccines may be able to overcome a pre-existing immunity to poxviruses due to previous smallpox vaccination (Belyakov, et al., (1999) Proc. Natl. Acad. Sci. U.S.A. 96:4512-7). The mucosa harbors dendritic cells, which are important targets for EBNA-1 vaccines and immunotherapy. Thus, mucosal vaccination strategies for both polypeptide and DNA vaccines are contemplated. While the mucosa can be targeted by local delivery of a vaccine, various strategies have been employed to deliver immunogenic proteins to the mucosa (these strategies include delivery of DNA vaccines as well, e.g., by using the specific mucosal targeting proteins as vector targeting proteins, or by delivering the vaccine vector in an admixture with the mucosal targeting protein).

For example, in a specific embodiment, the immunogenic polypeptide or vector vaccine can be administered in an admixture with, or as a conjugate or chimeric fusion protein with, cholera toxin, such as cholera toxin B or a cholera toxin A/B chimera (Hajishengallis (1995) J. Immunol. 154:4322-32; Jobling and Holmes (1992) Infect Immun. 60:4915-24). Mucosal vaccines based on use of the cholera toxin B subunit have been described (Lebens and Holmgren, (1994) Dev Biol Stand 82:215-27). In another embodiment, an admixture with heat labile enterotoxin (LT) can be prepared for mucosal vaccination.

Other mucosal immunization strategies include encapsulating the immunogen in microcapsules (U.S. Pat. No. 5,075,109, No. 5,820,883, and No. 5,853,763) and using an immunopotentiating membranous carrier (WO 98/0558). Immunogenicity of orally administered immune-gens can be enhanced by using red blood cells (rbc) or rbc ghosts (U.S. Pat. No. 5,643,577), or by using blue tongue antigen (U.S. Pat. No. 5,690,938). Systemic administration of a targeted immunogen can also produce mucosal immunization (see, U.S. Pat. No. 5,518,725).

### VII. Pharmaceutical Compositions and Administration

Pharmaceutical compositions according to the present invention., and for use in accordance with the present invention, may include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be by any convenient route for those of skill in the art, though is preferably by injection, e.g. cutaneous, subcutaneous or intra-dermal.

For injection, the active ingredient will be in the form of a parenterally acceptable aqueous solution which is pyrogen-free and has suitable pH, isotonicity and stability. Suitable diluents, which are pharmaceutically acceptable and may be preferred, have been discussed already above.

Oral administration may be used, in which case the pharmaceutical composition may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Vaccines may be administered by aerosol to the airways, using a suitable formulation, e.g. including particles of a size which travels to the appropriate parts of the airways. This may be a dried powder rather than aqueous suspension. The compositions of the present invention can be administered to any animal, including fish, amphibians, birds, and mammals (where mammals include, but are not limited to, monkeys, pigs, horses, cows, dogs, cats, and humans). The compositions may be administered via any suitable mode of administration, such as intramuscular, oral, subcutaneous, intradermal, intravaginal, rectal, or intranasal administration. The preferred modes of administration are oral, intravenous, subcutaneous, intramuscular or intradermal administration. The most preferred mode is parenteral, including subcutaneous administration.

The frequency of administration, including boosters if required, and other techniques associated with immunization are well known to those skilled in the art and if not already described or determined can be done so without undue experimentation. For example, the appropriate immunoprotective, non-toxic, and unique immune response-inducing amount of the composition of this invention may be in the range of the effective amounts of antigen in conventional vaccines. It will be understood however, that the specific dose level for any particular subject will depend upon a variety of factors including the age, general health, sex, and diet of the subject. Other factors influencing dose level include, but are not limited to, the time of administration, the route of administration, synergistic, additive, or antagonistic interactions with any other drugs being administered, and the amount of protection or the level of induction of the immune response being sought. For example, in a combination vaccine, the dosage of the vaccine of the present invention may need to be increased to offset the interference of the other vaccine components.

The compositions of the present invention, e.g., a therapeutic vaccine comprising the virus specific vaccine and an IL-10 antagonist, can be used in combination with other vaccines using methods well known to those skilled in the art. Viral vaccines include, but are not limited to, those against viruses or diseases such as hepatitis, Epstein Barr virus, human papilloma virus viruses, smallpox virus, HIV, chickenpox, mumps, and measles. Various regimens of exposure to a specific virus and subsequent administration of vaccines or combination vaccines are included and can be determined using methods well known to those skilled in the art, based on the disclosure provided herein

Mutant viruses or other agents that induce a viral specific Th1 immune response, can be administered along with a pharmaceutically acceptable carrier or diluent. Examples of such pharmaceutically acceptable carriers or diluents include water, phosphate buffered saline or sodium bicarbonate buffer. A number of other acceptable carriers or diluents are also known in the art.

### VIII. Kits

The invention is further directed to kits containing the vaccine against the virus and/or pharmaceutically acceptable composition thereof, and the antagonist of the immunosuppressive cytokine and/or a pharmaceutically acceptable composition thereof, as well as instructions for administration. In particular embodiments the vaccine and the neutralizing IL-10 or IL-10R antibody can be packaged separately or together. Furthermore, the kit may also comprise other biological agents.

The broad scope of this invention is best understood with reference to the following examples, which are not intended to limit the inventions to the specific embodiments.

### EXAMPLES

### I. General Methods.

Standard methods of biochemistry and molecular biology are described or referenced, e.g., in Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.. Standard methods also appear in Ausbel et al. (2001.) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al., (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protects in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protcols in Immunology, Vol. 4, John Wiley, Inc., New York.

Methods for flow cytometry, including fluorescence activated cell sorting detection systems (FACS®), are available, See. e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use. e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al., (2002) Basic Histology:Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc. Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742: Menne et al. (2000) Bioinformatics Applications Note 16:741-742; Wren et al. (2002) Comput. Methods Programs Biomed. 68:177-181: von Heijne (1983) Eur. J. Biochem. 133:17-21; von Heijne (1986) Nucleic Acids Res. 14:4683-4690.

### II. Mice and Virus

C57BL/6 mice were obtained from the Rodent Breeding Colony (The Scripps Research Institute, La Jolla, Calif.). The LCMV-GP₆₁₋₈₀-specific CD4+ TcR transgenic (SMARTA) mice and LCMV-GP₃₃₋₄₁-specific C8+ transgenic (P14) mice used, were described in Oxenius, et al. (1998) Eur. J. Immunol. 28:390-400. All mice were housed in pathogen-free conditions in accordance with NIH and IACUC guidelines.

Mice were infected intravenously with 2×10⁸ plaque forming units (PFUs) of LCMV-Arm or LCMV-CI 13. Virus stocks were prepared and viral titers were quantified as described in Borrow, et al. (1995) J. Virol. 69:1059-1070. All experiments contained 3-6 mice per group and were repeated minimum of 3 times.

### III. T Cell Isolation and Transfer

CD4 and CD8 T cells were purified from the spleens of naïve SMARTA and P14 mice, respectively, by negative selection (StemCell Technologies, Vancouver, BC). 1000 purified cells from each population were co-transferred i.v. into C57BL/6 mice. Each of these transgenic T cell population behave similarly to their endogenous (i.e. host derived) T cell counterparts based on tetramer analysis and intracellular cytokine staining (see, e.g., Wherry, et al. (2003) supra; and Borrow, et al (1995) J. Virol. 69:1059-1070). The number of SMARTA and P14 cells was determined by multiplying the frequency of Thy1+ cells, as determined by flow cytometry, by the total number of splenocytes.

### III. Quantitative PCR

RNA from total splenic mononuclear cells was obtained and amplified as described in Brooks et at. (2006) supra. RNA expression was normalized by input concentration and amplified by using the Qiagen ONE-STEP RT-PCR kits (Qiagen). The ASSAYS-ON-DEMAND Real-Time IL-10 expression kit (Applied Biosystems) as used to amplify IL-10 RNA. To quantify IL-10 RNA a standard curve was generated by 10-fold serial dilutions of total splenic RNA (1 μg to 1 pg total RNA, standard curve: r²>0.99) from CI 13 infected splenocytes and a relative number of IL-10 RNA determined. Amplifications were performed on the AB17700 amplifier (Applied Biosystems).

### IV. Intracellular Cytokine Analysis and Flow Cytometry

Splenocytes were stimulated for 5 hours with 5 μg/ml of the MHC class II restricted LCMV-GP₆₁₋₈₀ or 2 μg/ml of the MHC class I restricted LCMV-NP₃₉₆₋₄₀₄, GP₃₃₋₄₁, or GP₂₇₆₋₂₈₅ peptide (all 99% pure; Synpep.) in the presence of 50 U/ml recombinant murine IL-2 (R&D Systems and 1 mg/ml brefeldin A (Sigma). Ex vivo administration of IL-2 did not alter cytokine production. Cells were stained for surface expression of CD4 (clone RM4-5; Pharmingen) and CD8 (clone 53-6.7; Caltag). Cells were fixed, permeabilized and stained with antibodies to TNFα (clone MP6-XT22: ATCC), IFNγ (clone XMG1.2; ATCC) and IL-2 (clone JES6-5H4; ATCC). Flow cytometric analysis was performed using the Digital LSF II (Becton Dickinson). MHC class I and class II tetramers were produced as described in Homann, et al (2001) Nat. Med. 7:913-919. The absolute umber of virus-specific T cells as determined by multiplying the frequency of tetramer or IFNγ cells by total number of cells in the spleen.

V. In vivo IL-10R and PD-L1 Specific Antibody Treatment

C57BL/6 mice received 250 μg/mouse per intraperitoneal (i.p.) injection of IL-10R specific antibody (clone 1B1.3a; Schering-Plough) and/or 200 mg/mouse per i.p. injection of PD-L1. specific antibody (Harvard Medical School) beginning on day 25 or 30 after LCMV-CI 13 infection and continuing every three days for 5 treatments. Treatment with the rat IgG1 isotype control antibody (anti-E. coli b-galactosidase Mab, clone KM1 .GL113; Schering-Plough) had no effect on T cell, responses or viral replication.

### VI. DNA Vaccinations

Plasmid pCMV-GP encodes the entire LCMV glycoprotein. The parental control vector, pCMV does not contain LCMV sequences. Both pCMV-GP and parental pCMV vectors were described in Yokoyama, et al. (1995) J. Virol. 69:2684-2688. pCMV-GP encodes both CD4 and CD8 T cell LCMV glycoprotein epitopes, but not CD4 and CD8 T cell LCMV nucleoprotein epitopes. Plasmids were propagated in parallel in E. coli and purified using an endotoxin-free plasmid purification kit (Qiagen). C57BL/6 mice received DNA injection (bilateral 50 μl injections of plasmid DNA in saline (100 μg/mouse) into the anterior tibial muscles) on days 29 and 34 after LCMV-CI 13 infection.

### VII. Statistical Analysis

Student's t-tests were performed using SigmaStat 2.0 software (Systat Software, Inc.).

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of treating a chronic or persistent viral, infection comprising administering to a subject in need of treatment, an effective amount of a vaccine against a virus, wherein the virus is a causative agent for the persistent or chronic viral infection, in combination with an antagonist of an immunosuppressive cytokine.

2. The method of treatment of claim 1, wherein the combination of the vaccine against the virus and the antagonist of the immunosuppressive cytokine exhibits synergy in the treatment of the chronic or persistent viral infection.

3. The method of claim 1, wherein the immunosuppressive cytokine is IL-10.

4. The method of claim 1, wherein the antagonist of the immunosuppressive cytokine comprises a soluble IL-10 receptor (IL-10R) polypeptide.

5. The method of 4, wherein the soluble IL-10R polypeptide comprises a heterologous polypeptide.

6. The method of 5, wherein the heterologous polypeptide comprises an Fc portion of an antibody molecule.

7. The method of claim 4, wherein the soluble IL-10R polypeptide is pegylated.

8. The method of claim 1, wherein the antagonist of the immunosuppressive cytokine is a neutralizing IL-10 or IL-10 receptor (IL-10R) antibody or antibody fragment thereof.

9. The method of claim 8, wherein the neutralizing IE-10 or IL-10R antibody is a monoclonal antibody.

10. The method of claim 9, wherein the monoclonal antibody humanized or fully human.

11. The method of claim 8 wherein the antibody fragment is selected from the group consisting of a Fab, Fab2, Fv, and single chain antibody fragment.

12. The method of claim 1, wherein the chronic or persistent viral infection is selected from the group consisting of HBV, HCV, HIV, EBV, and LCMV.

13. The method of claim 1, wherein the vaccine is a DNA vaccine.

14. The method of claim 1, wherein the antagonist of the immunosuppressive cytokine is administered before the vaccine against the virus.

15. A pharmaceutical composition for use in the treatment of chronic or persistent viral infections comprising:
(a) a vaccine against a virus, wherein the virus is a causative agent for the persistent or chronic viral infection and a pharmaceutically acceptable carrier; and
(b) an antagonist of an immunosuppressive cytokine and a pharmaceutically acceptable carrier.

16. A kit comprising:
(a) a vaccine against a virus, wherein the virus is a causative agent for the persistent or chronic viral infection and a pharmaceutically acceptable carrier; and
(b) an antagonist of an immunosuppressive cytokine and a pharmaceutically acceptable carrier.

17. A method of treating a chronic or persistent viral infection comprising administering to a subject in need of treatment an effective amount of a vaccine against a virus, wherein the virus is a causative agent for the persistent or chronic viral infection, in combination with a neutralizing IL-10 or IL-10R antibody or antibody fragment thereof.

18. The method of treatment of claim 17, wherein the combination of the vaccine against the virus and the neutralizing IL-10 or IL-10R antibody or antibody fragment thereof exhibits synergy in the treatment of the chronic or persistent viral infection.

19. The method of claim 17, wherein the neutralizing IL-10 or IL-10R antibody is a monoclonal antibody.

20. The method of claim 19, wherein the monoclonal antibody is humanized or fully human.

21. The method of claim 17, wherein the vaccine is a DNA vaccine.

22. The method of claim 17, wherein administering the vaccine in combination with the neutralizing IL-10 or IL-10R antibody results in a 2-fold increase in virus specific CD8 T cells when compared to vaccine or antibody treatment atone.

23. The method of claim 17, wherein IFNγ producing virus specific T cells increase by 5-fold when compared to vaccine or antibody treatment alone.

24. The method of claim 17, wherein administering the vaccine in combination with the neutralizing IL-10 or IL-10R antibody results in a 24-fold decrease of viral titer compared to pretreatment levels.

25. The method of claim 17, wherein the neutralizing IL-10 or IL-10R antibody or antibody fragment thereof is administered before the vaccine against the virus.

26. A pharmaceutical composition for use in the treatment of chronic or persistent viral infections comprising:
(a) a vaccine against a virus, wherein the virus is a causative agent for the persistent or chronic viral infection and a pharmaceutically acceptable carrier; and
(b) a neutralizing IL-10 or IL-10R antibody or antibody fragment thereof and a pharmaceutically acceptable carrier.

27. A kit comprising:
(a) a vaccine against a virus, wherein the virus is a causative agent tor the persistent or chronic viral infection and a pharmaceutically acceptable carrier; and
(b) a neutralizing IL-10 or IL-10R antibody or antibody fragment thereof, and a pharmaceutically acceptable carrier.
